(19) United States
(12) Patent Application Publication
(10) Pub. No.: US 2005/0031702 A1
(43) Pub. Date: Feb. 10, 2005

(54) Application of arsenic as a cancer and heart disease prevention agent

(76) Inventor: Gary Michael Kayajanian, Arlington, VA (US)

Correspondence Address: GARY KAYAJANIAN, Ste. 1411, 1600 S. Joyce St., Arlington, VA, 22202, US

(21) Appl. No.: 10/634,869

(22) Filed: Aug. 6, 2003

(51) Int. Cl. A61K 33/00; C02F 1/42

(52) U.S. Cl. 424/600; 210/660

(57) ABSTRACT

Modest levels of arsenicals in drinking water (roughly 25-<75 μg/L or ppb) are associated with significant reductions of specified and/or total cancers in men and women and of heart disease in men.

### CROSS REFERENCE

Bac, I. -J. U.S. Pat. No. 6,309,672 (Oct. 30, 2001).

### FEDERALLY FUNDED RESEARCH AND DEVELOPMENT STATEMENT

This invention was not supported by federal funds.

### BACKGROUND OF THE INVENTION

This invention relates to the extended use of low exposures of arsenic in water-soluble form a a cancer- and heart disease-prevention-agent in humans. The body of scientific data supporting the cancer claim consists of three epidemiology data sets. The American data set also supports the heart disease claim in men.

In the common lore, arsenic, especially As₂O₃, is viewed as an acute poison, used at very high exposures by elderly women to kill visiting gentlemen in “Arsenic and Old Lace.” Forms of arsenic have found many uses as a killing agent (e.g., insecticide, fungicide, and pesticide), even though arsenicals have been “sold” with more gentle language (like, wood preservative). Arsenic has been the subject of regulatory review as a harmful contaminant in drinking water. At the high exposures found in the Himalayas and Andes, arsenic has been characterized as carcinogenic at multiple tissue sites. In 1976 under the Safe Drinking Water Act, the U.S. Environmental Protection Agency, (EPA) proposed an interim maximum contaminant level of 50 micrograms per liter (μg/L) as part of the National Interim Primary Drinking Water Standards. NRC (National Research Council) (1999). Arsenic in Drinking Water. (This inventor assumes American water systems all deliver water at or below 50 μg/L arsenic.) More recently, EPA Administrator Whitman affirmed Administrator Browner's purposeful but scientifically oblivious decision to lower that arsenic contaminant standard to 10 μg/L, relying on a strict monotonic extrapolation to zero exposure of a high dose lung plus bladder plus liver cancer mortality measure excess in the Taiwan data set.

The NRC Report cited above documents arsenic's high dose uses as treatment for a broad spectrum of symptoms and illnesses, especially in the later half of the nineteenth century; it also comments on the lack of data supporting the claim of “essentiality” for arsenic, even though it is found universally in living organisms.

A low dose cancer prevention claim for arsenic parallels an earlier claim made for dioxin by this inventor, but is clearly not derivative: dioxin is a specific cyclic organic chemical which binds preferentially to a pair of cell-produced agents; the resulting complex passes into the nucleus and binds to DNA, where it may act as a promoter blocker of cancers. The subject of this invention is the broad collection of arsenic molecules, for which no known binding system or any possible cancer prevention mechanism has been identified NRC (1999). There is no published evidence that arsenic binds to the Ah receptor and/or the Arnt protein, which are the components of the dioxin binding system. Kayajanian U.S. Pat. No. 6,444,698. The key to the beneficial effects attributed to arsenic in this invention boils down to a modest level of exposure to arsenicals (between 25-<75 i) in drinking water over an extended period of time. Lower and higher levels of arsenic exposure are harmful. Kayajanian, Ecotox. and Environ. Safety 55, 139-142 (2003). Any claims made by others for human treatments involving high dose arsenic exposures, as described below.

Bae U.S. Pat. No. 6,309,672 claims that arsenic hexoxide (As₄O₆), a polycyclic compound isolated and purified from a natural product or reproduced in the laboratory, at pharmaceutical doses is a treatment of malignant cancers sensitive to it—specifically human cancers of the uterus, lung, maxillary sinus, kidney and urinary bladder. In his human cancer treatment, the effective daily dose of arsenic hexoxide was 4 grams (for an average treatment of two years), approximately 40,000 times greater than the arsenic exposure of 50 ppb in drinking water called for in this invention: 4 grams daily for a Bae patient compared to 100 μg in two liters of water daily for an American. A significantly effective cancer prevention treatment in women and men, which Bae does not claim or demonstrate in humans, at less than 0.01% his arsenic hexoxide dosage for the arsenic compounds found in drinking water is sufficiently novel to justify a patent for this invention on the cancer claims. Bac explains his high dose experimental observations as apoptosis, a form of cell destruction.

Bae cites a paper by Chen et al. (May 1, 1997) in his references, published more than a year before his patent filing, which recites the use of arsenic trioxide (As₂O₃) as an acute treatment on acute promyelocytic leukemia cells. The treatment is effective over an exposure range of 0.5-2.0 μmoles per liter, but ineffective at 0.1 μmoles per liter: These values are equivalent to 0.1-0.4 μg/ml and 0.02 μg/ml, respectively. Chen requires just 9-36 percent of Bae's arsenic hexoxide dose to produce an acute effect on cells with arsenic trioxide. (Two percent of Bae's dose [0.1 μmoles per liter] is an ineffective cellular dose for arsenic trioxide.) For treating humans, Bae's prolonged daily exposure to arsenic hexoxide is 1818-times greater than his effective cellular dose and 40,000-times greater than 50 ppb. Chen reports no human exposure data, but if he treated cancers in humans with an 1818-times greater daily dose of arsenic trioxide, that exposure would be 3600-14,400-times greater than this invention's 50 ppb or Ig/L. (Even the ineffective dose would be 720-times greater than 50 ppb.)

Abstracts in Bae's references list arsenic trioxide or other arsenicals, usually in conjunction with other compounds, as apopotosic agents. What distinguishes this invention from Chen et al. and Bae's other references is the inherent safety attached to lifetime exposures of a 50 ppb level of arsenic in drinking water. After all, if arsenicals were as apoptosic by themselves as those papers and abstracts claim at the 50 ppb in drinking water, humans should not escape the first twenty years of life, when cell growth primarily occurs, without enormous, even greater levels of cell death. These other inventions rely on arsenicals as “-cide” agents, killing growing cells preferentially.

The heart disease claim made in this patent application, like the cancer claims, is based on a benefit that attaches to men exposed to arsenic in drinking water at daily exposures near 50 ppb.

### SUMMARY OF THE INVENTION

Arsenic is an elemental chemical. It and most of its salts are readily soluble in water. The measure of arsenic in drinking water is usually presented as parts per million (p parts per billion (ppb) or micrograms per liter (μg/L). EPA regulations for arsenic have employed the μg/L measure, which has ppb as a numeric equivalent. In the one American epidemiology data set examined for this invention, average daily arsenic intake ranged from 0-<175 ppb. In the Taiwan data set of 42 villages, also discussed below, villagers had calculated exposures ranging from 10 ppb to 934 ppb; one well had arsenic levels measured as high as 1,752 ppb. A number of published papers report on the level of total arsenic assayed in human blood, urine, hair and nails. NRC (1999).

The EPA regulators and the scientists (other than this inventor) who have examined high dose arsenic epidemiology studies have claimed arsenic is carcinogenic at multiple tissue sites, like lung, liver, bladder, kidney, colon and skin in both men and women. Chen, C. J., et al., Cancer Res. 45, 5895-5899 (1985). Also see, Morales, K. H., Ryan, L. M. et al., Environ. Health Perspect. 108, 0.655-661 (2000); Smith, A. H., et al., Science 296, 2145-2146 (2002); NRC (1999); and EPA, Report: Arsenic in Drinking Water Rule Economic Analysis (EPA 815-R-00-026) (2000). Underlying all the arsenic cancer characterization is the ingrained notion (supported only in part by data) that reducing exposure to this chemical, designated a carcinogen at high exposure, will result in a reduction of the cancer risk over the full range of exposures (i.e., from high exposure down to zero exposure. The inventor has reanalyzed available data from three epidemiology data sets, including the Taiwan data set EPA relied on to reduce the maximum allowable arsenic level in drinking water from 50 ppb to 10 ppb: all three demonstrate a J-shaped cancer mortality response to exposure—in the two data sets amenable to a numeric analysis, the cancer mortality trough is associated with an arsenic in drinking water level around 50 ppb (42-60 ppb in one data set, 25-<75 ppb in the other). Kayajanian, G., (2003).

The Taiwan data set, published at pp. 308-309 of the 1999 NRC Report, cited above, reports on lung, liver and bladder cancer mortality in men and women in 42 Taiwan villages. These deaths are associated with population-sizes presented as man- or woman-years. The arsenic exposure for all villagers in a village is determined by a direct current measurement of the arsenic level in the well or wells supplying each village. (When more than one water well serves a village, the arsenic level for each well is rank ordered with that of the others, and the arsenic level of the middle well is taken by the NRC to represent exposure; when an even number of wells supply a village, the average of the middle two wells measures the arsenic exposure of those villagers.) The cancer measure used for the men and women in each village is lung+liver+bladder+cancer deaths per thousand person years. For the five villages reporting arsenic levels near 50 ppb (4260 ppb), the former regulatory standard, the cancer measures are 0.53 for men and 0.51 for women. For the five villages with lower arsenic levels (10-32 ppb), the cancer measures are significantly greater (p<0.001 for each sex): 1.65 for men and 1.62 for women Of course, the cancer measure gradually increases for villagers with arsenic levels above 60 ppb—to a peak of 2.43 for men and 1.99 for women at an arsenic exposure range of 650-698 ppb, thus accounting for the J-shape asserted above.

The American data-set was collected and published on by EPA scientists, who reported on sources of mortality in Millard County, Utah and the rest of the state. Lewis, D. R., et al., Environ. Health Perspect. 107, 359-365 (1999). Lewis et al. compared mortality in Millard County with that in the rest of the state. This inventor sought to avoid the issues of confounding inherent in the Lewis et al. paper, choosing to compare residents of Millard County with each other, grouping each sex by mean daily arsenic exposure in water from unpublished Agency data provided by request. Total cancer mortality is the endpoint and total cancer mortality per 100 people is the cancer measure. The cancer measure for men is about 10% lower (7.220 versus 8.047, not significant) for men with a mean arsenic exposure between 25-<75 ppb compared to 0-<25 ppb. The cancer measure is 65% lower (2.784 versus 9.189, p<0.000001) for women with a mean exposure between 25-<75 ppb compared to 0-<25 ppb. At the next higher arsenic exposure range (75-4125 ppb), the total cancer measure increases both for men (1.355, p<0.08) and women (9.504, p<0.01), thus accounting for the J-shape asserted above. Chen et al. (1985) associated high dose exposures of arsenic in men and women with significant increases in lung, liver, bladder, kidney, and skin and colon cancer mortality. When the sum of these cancers becomes the cancer measure for women, a greater (76%) but a less significant (p<0.01) reduction in mortality is associated with a 25-<75 ppb arsenic exposure in drinking water compared to 0-<25. See, Kayajanian, G. (2003). Even for the remaining cancers in women (i.e., total cancers except for lung, liver, bladder, kidney, skin and colon), a lower (67%) but still significant (p<0.0001) reduction in cancer mortality is associated with a 25-<75 ppb arsenic exposure in drinking water compared to 0-<25. Calculated from data in Kayajanian, 2003. The total cancer benefit in women associated with arsenic exposures from 25-<75 ppb occurs throughout life, but is most significantly evident from ages 60-79. Kayajanian, unpublished.

According to the NIH's SEER Cancer Statistics Review: 1973-1990 (1993), there are 570,000 cancers cases and 249,000 cancer deaths annually among women. If arsenic levels in drinking water are currently just barely in compliance with the old 50 ppb standard, lowering the arsenic level to 10 ppb should result in an annual increase of 1,087,000 cancers and 475,000 cancer deaths. This equates to 2979 extra cancers and 1301 extra cancer deaths per day, when the Millard County cancer data are applied nationally. If arsenic levels are compliant with the 10 ppb standard (or <25 pb), raising that level to 25-<75 ppb should result in an annual decrease of 373,000 cancers and 163,000 cancer deaths. This equates to 1024 fewer cancers and 447 fewer cancer deaths per day, when the Millard county cancer data are applied nationally. As a practical matter, most water systems currently deliver water to most Americans with arsenic levels below 10 ppb. So the greater benefit of this invention to the population would appear as a cancer or cancer mortality reduction, rather than an avoidance of a cancer or cancer mortality increase.

The Utah data set also offers numbers on mortality from other disease causes, including heart disease. Heart disease deaths are significantly reduced (31.66 versus 42.33 per 100 deaths, p<0.03), for men with a mean exposure between 25-<75 ppb compared to 0-<25 ppb.

According to the NIH's National Heart Lung Blood Institute Morbidity and Mortality Chartbook (May, 2000), there are 311,000 heart disease deaths annually in men. If arsenic levels in drinking water are currently just barely in compliance with the old 50 ppb standard, lowering the arsenic level to 10 ppb should result in an annual increase of 104,000 heart disease deaths. This equates to 287 extra heart disease deaths per day, when the Millard County heart disease deaths are applied nationally. If arsenic levels are compliant with the 10 ppb standard (or <25 ppb), raising that level to 25-<75 ppb should result in an annual decrease of 78,000 heart disease deaths. This equates to 214 fewer heart disease deaths per day, when the Millard heart disease data are applied nationally.

The third data set, from the 1982 Cuzick et al. article in the British Journal Cancer (45, 904-911, especially Table 3) is structurally different from the Taiwan and Utah data sets. The pooled population of men and women were intentionally dosed with arsenic for medicinal purposes and standardized cancer mortality was presented as a function of two variables: increasing total dose and time in years since the first dose. Here, unlike Taiwan and Utah, the outside reference serves as the lowest exposure group and the lowest dosed group (<500 mg) serves as the next lowest exposure group. Over all time intervals 10 cancers were observed in the lowest dosed group and 18.34 were expected from the outside reference (p<0.06). At the higher dosings, 14.41 cancers were expected, 24 were observed.

The Cuzick data also may be helpful in suggesting the effect of arsenic on cancer mortality. In the five years following the initial arsenic medication, 6.45 cancers deaths were expected; in the second five years, 6.67—a total of 13.12 cancer deaths. Over those ten years, 13 cancer deaths were recorded: but only 2 occurred in the first five years and 11 occurred in the second five years (p<0.02)—a timing which suggests additionally that, irrespective of exposure level, arsenic delays cancer deaths.

A more detailed discussion of the Taiwan, Utah and Cuzick data sets are found in Kayajanian (2003), referenced above. That paper and other papers and reports cited in this patent are incorporated herein.

Methods for assaying the arsenic level in drinking water over the range of interest for this invention are evidenced in the Taiwan and Utah data sets. Arsenic's use as a medicinal (Fowler's aqueous solution in Cuzick et al.) attests to a method to purify and concentrate arsenic from one arsenic source to be added to waters with less than 25 ppb arsenic (even waters with less than 50 ppb arsenic) to raise the level of the treated water to approximately 50 ppb. Alternatively, natural water sources with high arsenic levels could be mixed with waters with less than 25 ppb arsenic (even waters with less than 50 ppb arsenic) to raise the level of the mixed waters to around 50 ppb.

### DESCRIPTION OF THE PREFERRED EMBODIMENTS

There are a number of attributes associated with this invention:

(1) Daily arsenic exposure in drinking water between 25-<75 μg/L is associated with a significant (p<0.000001) 65% reduction of total cancer deaths in women compared to women exposed to 0-<25 μg/L. (American data set.)

(2) Daily arsenic exposure in drinking water between 25-<75 μg/L is associated with a significant (p<0.03) 25% reduction of total heart disease deaths in men compared to men exposed to 0-<25 μg/L. (American data set.)

(3) Liver and bladder cancers are significantly reduced in men and in women in villages with 42-60 μg/L daily arsenic exposure compared to those in villages with 10-32 μg/L. (Taiwan data set.)

(4) Following full implementation, current regulatory policy, which compels water systems to lower or maintain arsenic levels at or below 10 μg/L, is expected to result in 1024 extra cancers diagnosed and 447 extra cancer deaths per day in women and 214 extra heart disease deaths in men compared to a policy which compels an arsenic level in drinking water between 25-<75 μg/L.

(5) For some segments of the population, arsenic levels in water can be adjusted to 25-<75 μg/L by adding inorganic arsenic, or mixing water from multiple sources.

(6) For some segments of the population with an arsenic level currently between 25-<75 μg/L, not complying with the not-yet-fully-implemented 10 μg/L standard is the best “adjustment”.

(7) For other segments of the population exposed to, water with an arsenic level below 25 μg/L, individuals may choose to add arsenic to, the diet as a supplement.

1. A method for reducing total cancer morbidity and mortality in women by adjusting the arsenic level in drinking water to 25-<75 μg/L.

2. A method according to claim 1 when the cancer is lung cancer.

3. A method according to claim 1 when the cancer is liver cancer.

4. A method according to claim 1 when the cancer is bladder cancer.

5. A method according to claim 1 when the cancer is kidney cancer.

6. A method according to claim 1 when the cancer is skin cancer.

7. A method according to claim 1 when the cancer is colon cancer.

8. A method for reducing cancer mortality in men by adjusting the arsenic level in drinking water to 42-60 μg/L.

9. A method according to claim 8 when the cancer is lung cancer.

10. A method according to claim 8 when the cancer is liver cancer.

11. A method according to claim 8 when the cancer is bladder cancer.

12. A method for reducing heart disease mortality in men by adjusting the arsenic level in drinking water to 25-<75 μg/L.

13. A method for not increasing total cancer morbidity and mortality in humans by maintaining the arsenic level in drinking water between 25-<75 μg/L even though such maintenance would violate the newly enacted 10 g upper bound EPA standard.

14. A method according to claim 13 when the cancer is lung cancer.

15. A method according to claim 13 when the cancer is liver cancer.

16. A method according to claim 13 when the cancer is bladder cancer.

17. A method according to claim 13 when the cancer is kidney cancer.

18. A method according to claim 13 when the cancer is skin cancer.

19. A method according to claim 13 when the cancer is colon cancer.

20. A method for not increasing heart disease mortality in men by maintaining the arsenic level in drinking water between 25-<75 μg/L even though such maintenance would violate the newly enacted 10 μg/L upper bound EPA standard.
